(19) United States
(12) Patent Application Publication
(10) Pub. No.: US 2022/0151278 A1
(43) Pub. Date: May 19, 2022

(54) Dietary Supplement

(71) Applicant: Gratuk Technologies PTY LTD, New South Wales (AU)

(72) Inventor: Malcolm Ball, New South Wales (AU)

(21) Appl. No.: 16/920,272

(22) Filed: Jul. 2, 2020

Related U.S. Application Data
(63) Continuation of application No. 14/383,183, filed on Sep. 5, 2014, now abandoned.

(30) Foreign Application Priority Data
Mar. 5, 2012 (AU) 2012900900

(51) Int. Cl. A23L 33/22; C08L 97/02; A61K 36/899; A23K 20/163

(52) U.S. Cl. CPC A23L 33/22; C08L 97/02; C08L 2205/16; A23K 20/163; A61K 36/899

(57) ABSTRACT

Use of a dietary fibre material extracted from sugar cane in the manufacture of a food product that is formulated to ameliorate the effects of diabetes.

### CROSS REFERENCE TO RELATED APPLICATION

This application is a continuation application of U.S. patent application Ser. No. 14/383,183, filed Sep. 5, 2014, which is a national stage application under 35 U.S.C. 371 of PCT Application No. PCT/AU2013/000201 having an international filing date of Mar. 5, 2013, which designated the United States, which PCT application claimed the benefit of Australian application No. 2012900900, filed Mar. 5, 2012, all of which are incorporated by reference in their entirety.

### TECHNICAL FIELD

The invention relates to the field of commercial food supplement manufacture. In particular, the invention relates to a dietary supplement, the use of said supplement in the diet of an individual, and the method of manufacture of said supplement.

### BACKGROUND OF THE INVENTION

Diabetes is becoming more prevalent in the human populations of the world. Although all three types of diabetes (Mellitus, insipidus, gestational) occur in humans, only mellitus and gestational are related to insulin and blood glucose levels.

Diabetes Mellitus results when the body loses the ability to regulate blood glucose due to either loss of insulin production by the pancreas (Type 1) or insensitivity to insulin produced by the body (Type 2). In many individuals Type 1 and Type 2 diabetes occurs simultaneously; more recently the term Adult Latent Autoimmune Diabetes has been used to describe Diabetes Mellitus that has components of Type 1 and Type 2 in the adult population.

In the case of gestational diabetes a pregnant woman loses the ability to regulate blood glucose due to hormone imbalances caused by the gestating foetus. Normal regulation is usually regained after the birth of the child, however the condition needs to be managed while the individual is pregnant and severe complications do occasionally occur. Additionally, gestational diabetes can results in a number of negative effects on the child such as a pre disposition to Type 2 diabetes mellitus later in life. It is becoming accepted that gestational diabetes has a significant relationship to nutrition, and it has been suggested that many cases of gestational diabetes may be treated with nutritional supplements.

The causes of diabetes are not well understood but the most common risk factors referred to in the literature are obesity, hyperlipemia (caused by high fat diets), genetic predisposition and autoimmune conditions.

Diabetes is treated in one of three ways depending on the type and severity of each case. Diet control, is used for those in the high risk group and mild sufferers; oral medications are available for intermediate cases; and sub-cutaneous injection of recombinant insulin is used for the most severe cases (as Type 1 diabetes is caused by the loss of insulin production insulin replacement therapy is almost always used).

In the years of 2007-2008 the number of diagnosed cases of diabetes mellitus in Australia was recorded as 898,800 approximately 90% of those cases being Type-2 diabetes. It has also been observed that gender, regional and socioeconomic factors are not important; although age does seem to be a factor (96% of sufferers were over 35 years of age).

As obesity is a major contributing factor and the population is ageing these figures are expected to rise over years to come. Treatment of the disease is a major drain on public and private health resources, and it can be complicated to find a treatment regime that works for each individual any method of reducing risk and/or alleviating symptoms is of significant value to sufferers and to the broader population.

A compounding factor in the treatment of diabetes is that diabetics have been found to have a higher incidence of other chronic conditions than the general population. This means that the presence of potential food allergies play a major role in the selection of diet control for diabetic individuals. Common food allergies and intolerances of diabetics have been reported with respect to wheat, dairy, soy, and oats (amongst others). It has been estimated that 2% of the general population suffers from food allergies but diabetics have an incidence of allergies up to 40% higher than that. It is now well accepted that medication alone is insufficient in treating diabetes and full lifestyle regimes are routinely prescribed in conjunction with medication, both oral and injected. Large scale cohort studies of multiple ethnicities and lifestyles have shown that regardless of other factors (such as genetic predisposition and body weight) a “healthy” diet has a statistically significant improvement on outcomes both for diabetes mellitus itself and secondary correlating diseases.

The proportion of diabetics suffering food allergies is therefore significant, and a hypoallergenic treatment would be preferred. Currently diet related treatments for diabetes involve high fibre foods, however this fibre is most often derived from wheat and/or oats. It is also known that not all high fibre diets work to control blood glucose level. Recently the veracity of the claims that high fibre is important to blood glucose control has been questioned. It has been suggested by some that this discrepancy has arisen because highly refined fibres that are essentially without additional nutritional benefit do not confer protective effects or benefits and a “whole plant” or “total dietary fibre” in needed. Total dietary fibres have soluble and insoluble components and are from sources that contain micronutrients such as antioxidants. Accordingly regulatory and medical bodies are in the process of reviewing legislation and terminology in regards to so called “high fibre” diets.

Accordingly, it is an object of the invention to improve the delivery of a high quality total dietary fibre to the diet of individuals that are at risk of diabetic conditions, that ameliorates the identified disadvantages of the prior art.

### SUMMARY OF THE INVENTION

According to one aspect of the invention, there is provided the use of dietary fibre material extracted from sugar cane in the manufacture of a food product that is formulated to ameliorate the effects of diabetic conditions.

Preferably, the sugar cane fibre is prepared via a process including the steps of: subjecting the sugar cane material to at least one wet diffusion step to separate sugars from a residual fibre material whilst maintaining nutrient content; and subjecting the residual fibre material to a rapid, low-heat drying process thereby to retain the biologically active molecules in the fibre, and to enhance the water retention properties of said residual fibre product.

There are a number of advantages to using dietary fibre material extracted from sugar cane in the way described above. Firstly, no adverse allergic effects have ever been recorded with this source. Also, this fibre source has been shown to improve gut lining health over and above other sources of fibre. It contains benefits of both soluble and insoluble fibre and has a ratio of fibres that more accurately represents natural foods than other products. It is also high in other micronutrients such as iron and has the ability to protect antioxidants.

Fibres separated from grasses such as sugarcane have several advantageous properties compared to incomplete (not whole plant fibres) such as bran, psyllium husk and inulin. The fibre is a true lignose, hemicellulose and cellulose combination such as the total dietary fibres found in most vegetables. Additionally even though sugarcane fibre is classed as almost entirely insoluble fibre, using the standard chemical methods of classification, it has many of the properties of soluble fibres as well such as it has a high water binding capacity (up to 8-10 times by weight) and a probiotic effect. Also even though insoluble fibres are known to have little or no effect on blood glucose levels it has been observed that, when prepared correctly, sugarcane fibre can have profound benefits on postprandial blood glucose levels. This is most likely a combination of the fact that the hemicellulose fraction of the fibre has soluble components that are released during digestion and that when prepared correctly the fibre retains a number of biologically active molecules.

This fibre not only has the effect of reducing postprandial blood glucose levels and lowering the Glycaemic Index (GI) of foods, but when prepared and formulated correctly can be used to produce foods that have continued beneficial effects on fasting glucose levels and a long term reduction in hyperglycaemia related complications. The beneficial effect of this invention is not limited to the reduction of high blood glucose levels; it may also be used to reduce the risk of hypoglycaemia by being included in energy rich foods for improved overall control of BGLs in diabetic and pre-diabetic individuals.

In addition, when this fibre source is prepared via the process as described herein, the fibre tends to retain its functionality with respect to diabetes to a greater level, due to the retention of biologically active molecules in the fibre. The fibre source also provides the correct dietary fibre level to address this condition in the majority of the population.

The invention also allows more flexible product formats to be developed, in particular that allow individuals suffering diabetic conditions to address to deficiency in their own way, especially when provided with the correct type of fibre in a relatively easy-to-use format. Individuals no longer have to rely on food manufacturers to generate high fibre foods that they can eat.

Preferably, the wet extraction step is a diffusion extraction, done under relatively low-shear conditions. The optimal wet extraction step temperature is in the range 25° C. to 70° C.

According to another aspect of the invention, there is provided a food product formulated to ameliorate the effects of diabetes; said food product containing dietary fibre material extracted from sugar cane, said dietary fibre material preferably having been prepared according to the steps defined above.

According to another aspect of the invention, there is provided a method of treatment of the effects of diabetes in an individual by feeding to said individual a food product incorporating dietary fibre material extracted from sugar cane; said dietary fibre material preferably having been prepared according to the method defined above.

Now will be described, by way of particular, non-limiting examples, preferred embodiments of the invention.

### BRIEF DESCRIPTION OF THE DRAWINGS

FIG. 1 presents average blood glucose data obtained using a food product of the present invention;

FIG. 2 presents data disclosing blood levels determined before and after the introduction of a food product of the present invention into a subject's diet; and

FIG. 3 presents change in blood glucose data over time obtained using a food product of the present invention

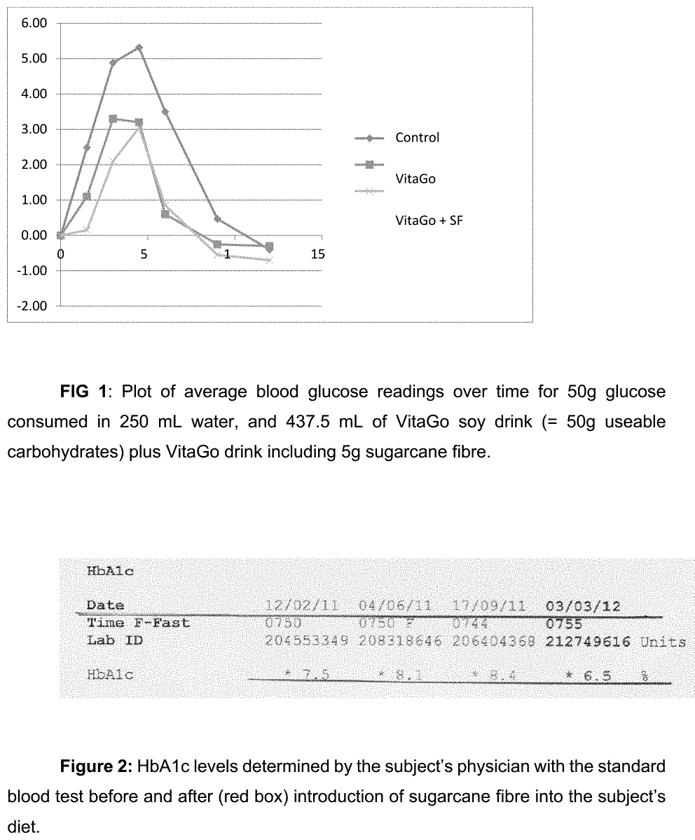

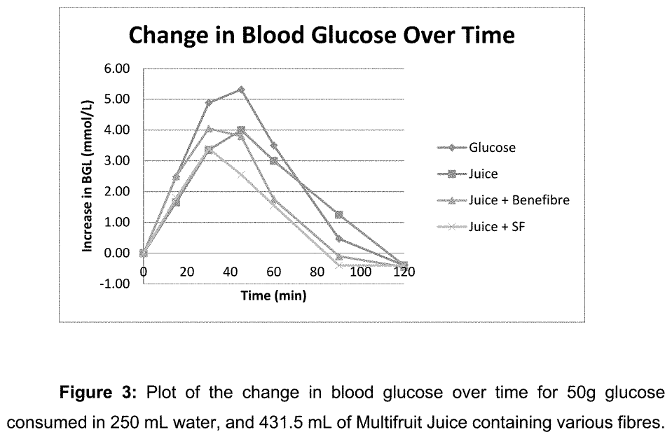

### DETAILED DESCRIPTION OF THE INVENTION

The current invention takes advantage of the properties of a dietary fibre isolate produced from sugar cane, in such a way that maximised retention and minimal destruction of the bioactive molecules occurs.

The method of preparation of the fibre material from sugar cane is broadly similar to that described in WIPO patent document no. WO2011/035381 by KFSU Pty Ltd, which is incorporated herein by reference. However, the process according to the present invention may be defined as having the following essential features:
1. A sugar cane size reduction step;
2. A relatively ‘gentle’ aqueous extraction stage that separates the fibre from other sugar cane fractions, including the sugar fraction, without causing degradation of the fibre functionality; and
3. A relatively gentle drying step that minimises degradation of the fibre functionality.

It is preferred that the extraction step be an aqueous diffusion extraction performed at a relatively neutral pH. It is also preferred that the drying step be a rapid vortex drying operation that, as may be achieved via a low temperature, vortex dryer, such as that supplied by Tensei in Japan (www.tensei-j.com).

It is understood that adequate dietary fibre is important to the healthy function of the digestive system. It is also known that dietary fibre levels can have an influence on the likelihood of development of diabetic conditions in humans. It is also thought that human diets tend to be deficient in dietary fibre, and/or use fibre sources known to cause allergies and intolerances, such as wheat and oats.

It is also understood that many fibre sources that are processed from grains and other ‘incomplete’ (i.e. not ‘whole plant’) sources do not significantly aid in the dietary control of blood glucose levels. It is further understood that many sources of fibre that were thought to have a beneficial effect have been shown in recent studies to either be ineffective or to only provide short term benefits.

It has also been shown that in many cases extraction and purification of micronutrients that have been shown to have a beneficial effect on diabetes either lose their benefit or have those benefits greatly reduced. It is thought that this is due to loss of essential co-factors and synergistic effects. The process described herein is therefore designed to maintain these micronutrients in a form that maintains high activity levels.

The invention provides for the use of sugarcane fibre in the formulation of foods or diets that seek to reduce the risk of development of diabetic conditions, or which ameliorate the symptoms of those conditions, if acquired. When prepared according to the invention, this fibre source, and the foods incorporating it, has a number of advantages over other fibre sources and food, including that:
It is relatively hypoallergenic;
It contains both insoluble and soluble fibre in beneficial proportions for dietary intake;
It contains a number of bioactive molecules that beneficially affect blood glucose levels and intestinal health to a greater degree than other fibre sources;
It can be prepared in a ‘chemical-free’ manner and contain no harmful trace elements, unlike fibre from other sources such as chemically modified starch;
It can be prepared in such a way as to retain the micronutrients and active molecules found in the “molasses” component of sugarcane, without the need to extract and purify those components for their biological function;
Chromium, polyphenols and certain high quality dietary fibres have all been shown to aid in lowering the GI of foods and improving insulin sensitivity. This product combines all three in a natural food that has been processed in a manner that retains beneficial effects and allows for synergistic action;
Other inventions have sought to isolate the various components however the combined effects of the components exceed that of individual extracts. Additionally the “whole food” nature of the products limits side effects and protects from overdose;

It is also known that too much fibre in the diet can have several negative side effects including but not limited to constipation, diarrhoea and bad flatulence. In one embodiment, where the fibre product is added as a supplement to an individual's diet, dietary fibre intake can be more easily controlled.

The supplement is also classed as a natural food which is increasingly important to many consumers.

The embodiments of the invention can take a number of forms, each with several advantages for users.

In this document:
A ‘carrier’ is a palatable substrate for the sugarcane fibre, which may or may not contain protein or other nutrients; including but not limited to: fruit extracts, broths, purees, dairy products, baked goods; and which may be in solid or liquid form.
‘Inert filler’ is any product used to increase the bulk size of fibre according to the invention to allow for ease of handling by the user. The filler may contain flavours or nutrients, and other dietary fibres to improve mouth feel, but does not necessarily contribute to the total benefit provided by the invention.
‘Pellet’ includes any compact form of the invention, including but not limited to:
   A dried pill or tablet in the manner of a vitamin.
   A soft lolly style lozenge that may be used as a treat or as an addition to other foods

All of the examples below can optionally be formulated with additional vitamins and bioactive molecules, or sweeteners such as stevia. Preferably any added nutrients would be sourced from natural ingredient to that a “natural” descriptor may be maintained for the final product.

### Example 1

In this example, 0.5-2.0 g of the active fibre is added to a flavouring medium and pressed into a pellet. Each pellet contains sufficient fibre and bioactives to help manage blood glucose levels. The pellets are prepared at a formulation level such that the dose may be varied if the consumer has been placed on a high fibre diet by their physician. However, if there is no other dietary control, the nature of the product means the dose may be increased without negative effects. The pellet may be taken during or immediately before or after a meal.

### Example 2

In this example the active fibre is mixed with a flavoured drink (for example fruit juice or milk) and pasteurised for sterility (1-5 g per 100-250 ml). A drink prepared in this manner is a convenient, ready-to-consume product to be taken with meals.

### Example 3

In this example the supplement is prepared as an easy-to-measure powder with flavours, stabilisers and an inert filler, formulated specifically to be combined with water. Specifically, the active fibre could be mixed with a dry flavour component and an inert filler to form easy-to-use granules. The dose (1-5 g) would be in a convenient single-serve sachet or in a multi-dose bulk pack. This example is best suited to aid weight loss (a major confounding factor in diabetes) as the granules can be mixed with water (thereby allowing less food to be consumed each meal).

### Example 4

In this example the supplement is prepared in a solid flavoured meal such as a biscuit or a bar (1-5 g per ready mixed food). Multiple biscuits can be consumed by an individual to provide a specific dosing regimen as needed for their lifestyle. This has two advantages over other delivery systems in that it feels more like a treat for the consume, and it eliminates the need for liquid, which is a concern for older diabetics that have bladder control issues. Specifically these foods can take 2 forms either:
1) The biscuit can be prepared without significant carbohydrate and sugars. This form would be used as a compliment to a meal to provide a dose without affecting blood sugar levels in its own right.
2) The biscuit is formulated with a defined sugar and carbohydrate dose to provide a combination of instant and long term energy for use as a replacement for high glucose “lollies” in the prevention of hypoglycaemia. This form is preferable to consumption of sweets as these often lead to hyperglycaemia which upsets the insulin cycle of the individual. The addition of the sugarcane fibre results in a sustained but smaller intensity increase in the blood glucose levels which is a preferable outcome.

### Example 5

In this example the fibre material is supplied as an ingredient for other manufacturers of high-fibre foods for the diabetic and/or weight control markets. This example provides several benefits for potential food manufacturers/suppliers:
If the fibre material is used to replace allergenic fibres such as wheat or psyllium husks then the product may be labelled as hypo-allergenic.
The fibre material supports the use of “all natural” marketing claims for the foods.
The fibre material provides other health benefits compared with other fibre sources, allowing the food manufacturer to potentially make more substantive claims.

The water retention capacity of the sugar cane, prepared as described above, is far greater than most commercial fibre sources. By using the active fibre in foods the manufacturer can reduce calorific content per kg of food. This may also result in a significant commercial saving for the manufacturer.

Food products and methods according to the invention make use of the unique qualities of cane based crops, particularly whole sugarcane, that have been prepared using a chemical-free, low-heat procedure. This makes it easy and convenient to use while still retaining the beneficial nutrients and bioactive molecules in the food.

According one particular feature of the invention, a sugar cane fibre material may undergo a pressure heating step at a pressure in the range of 100 psi to 140 psi.

The products and methods address several problems associated with poor fibre consumption, as well as having a positive impact on diabetic conditions, while also contributing to the elimination of the potential problems of intolerance and malabsorption in individuals that suffer allergies or intolerances to common fibre sources. The product may also be classed as a natural, whole food; meaning it does not have some of the problems associated with many pharmaceutical treatments, including some negative side effects.

### Example 6

To determine if addition of sugarcane fibre to liquid meal drinks can effectively reduce the GI, a study was carried out on a number of subjects. The method adapted from Sydney University G.I. standard methodology. The test was performed in the morning prior to eating or exercise.

Glucose (50 g) was dissolved into water (250 mL) immediately prior to consumption for use as a control. A commercially available breakfast drink—Vitasoy VitaGo variety (437.5 mL; equal to 50 g useable carbohydrate) with and without 5 g added sugarcane fibre (said fibre prepared according to the above described process) was consumed in less than 10 minutes.

Blood glucose levels (BGL) were measured using an Accu-Chek commercial diabetes glucose monitor and provided strips (strips batch number 698 expiry date 30 Apr. 2013). Baseline BGL was taken immediately prior to consuming the meal (t=0) by finger prick using the included lancing device. BGL levels were measured using the above method at t=15, 30, 45, 60, 90, and 120 minutes.

When multiple measurements were taken at the same time point (droplet used, wipe clean and then new droplet used) to determine the error range for the device.

Area under the curve was calculated using the trapezoidal method in Excel.

When the VitaGo without sugarcane fibre was consumed, BGLs had returned to approximately t=0 levels by 90 minutes, but there was a small spike in BGL at 120 minutes. Area under curve=136.9 mmol/L/minute, which was 49.4% of the control.

The average Coefficient of Variation (CoV) in the results was low at 3.67%. The 15 minute result had the highest CoV at 8.70%, however all others were close to or within the allowed range for the Australian standard for GI determination.

The sugarcane mixed with the VitaGo did not seem to case separation on the product. Separation had been observed with some soy based drinks in the past.

BGL had dropped below t=0 levels by 90 minutes, when the VitaGo plus sugarcane fibre was consumed, and remained consistent until 120 minutes. Area under curve=71.6 mmol/L/minute, which was 25.9% of the control. The average CoV in the results was very low at 1.62% and none of the values varied more than 4%.

From these results, we conclude that even in a product that already has a low GI, sugarcane fibre appears to have a marked effect in further reducing GI.
Control
VitaGo
VitaGo+SF

TABLE 1
Measured blood glucose levels at various times after
consumption of 437.5 mL VitaGo meal drink showing
averages and variation between the results.

| | B.G. | B.G. | B.G. | | |
|---|---|---|---|---|---|
| Time | (mmol/L) | (mmol/L) | (mmol/L) | | |
| (min) | (reading 1) | (reading 2) | (Ave) | StDev | CoV |
| 0 | 5.5 | 5.2 | 5.35 | 0.212132 | 3.97% |
| 15 | 6.1 | 6.9 | 6.50 | 0.565685 | 8.70% |
| 30 | 9.5 | 9.1 | 9.30 | 0.282843 | 3.04% |
| 45 | 7.8 | 8.3 | 8.05 | 0.353553 | 4.39% |
| 60 | 6.7 | 6.3 | 6.50 | 0.282843 | 4.35% |
| 90 | 5 | 5 | 5.00 | 0 | 0.00% |
| 120 | 5.6 | 5.7 | 5.65 | 0.070711 | 1.25% |

TABLE 2
Measured blood glucose levels at various times after consumption
of 437.5 mL VitaGo + 5 g sugarcane fibre meal drink
showing averages and variation between the results.

| | B.G. | B.G. | B.G. | | |
|---|---|---|---|---|---|
| Time | (mmol/L) | (mmol/L) | (mmol/L) | | |
| (min) | (reading 1) | (reading 2) | (Ave) | StDev | CoV |
| 0 | 6.1 | 6 | 6.05 | 0.070711 | 1.17% |
| 15 | 6.2 | 6.2 | 6.20 | 0 | 0.00% |
| 30 | 8.3 | 8 | 8.15 | 0.212132 | 2.60% |
| 45 | 9 | 9.2 | 9.10 | 0.141421 | 1.55% |
| 60 | 6.8 | 7 | 6.90 | 0.141421 | 2.05% |
| 90 | 5.5 | 5.5 | 5.50 | 0 | 0.00% |
| 120 | 5.5 | 5.2 | 5.35 | 0.212132 | 3.97% |

TABLE 3
comparison of the average BGL readings for glucose
control, 437.5 mL VitaGo meal drink, and 437.5
mL VitaGo meal drink + 5 g sugarcane fibre.

| Time | | | VitaGo + 5 g |
|---|---|---|---|
| (min) | Control | VitaGo | sugarcane fibre |
| 0 | 0.00 | 0.00 | 0.00 |
| 15 | 2.48 | 1.15 | 0.15 |
| 30 | 4.88 | 3.95 | 2.10 |
| 45 | 5.32 | 2.70 | 3.05 |
| 60 | 3.50 | 1.15 | 0.85 |
| 90 | 0.47 | −0.35 | −0.55 |
| 120 | −0.40 | 0.30 | −0.70 |

TABLE 4
Area under the curve calculations for blood glucose levels at various
times after consumption of glucose control, 437.5 mL VitaGo meal
drink, and 437.5 mL VitaGo meal drink + 5 g sugarcane fibre.

| Time | | VitaGo | Vita Go drink + 5 g |
|---|---|---|---|
| (min) | Control | drink | sugarcane fibre |
| 0 | | | |
| 15 | 18.625 | 8.625 | 1.125 |
| 30 | 55.25 | 38.25 | 16.875 |
| 45 | 76.5 | 49.875 | 38.625 |
| 60 | 66.125 | 28.875 | 29.25 |
| 90 | 59.5 | 12 | 4.5 |
| 120 | 1 | −0.75 | −18.75 |
| Total | 277 | 136.875 | 71.625 |
| GI | 100 | 49.4 | 25.9 |

### Example 7

The acute and long term benefits of the consumption of sugarcane fibre on the blood glucose levels of a diabetic individual were measured. The particular aim of the study was to determine if addition of sugarcane fibre to the diet of a sufferer of Type 2 Diabetes Mellitus improves the subject's health outcomes.

Insulin injections and blood glucose levels were measured as per standard suggested requirements for the individual. Average readings for breakfast and lunch (subject did not record values for dinner) were calculated from log book entries in the 6 months prior to commencement of sugarcane fibre therapy. Note was taken of both blood glucose levels and amount of insulin injected. Values below 4 mmol/L (representing a hypoglycaemic episode) were excluded from the calculations. Fourteen days of values were used for average calculations

Subject commenced consumption of 4 g of sugarcane fibre with the breakfast meal. Fibre was mixed in with breakfast of consumed mixed with juice at breakfast. Diet and exercise regimen were kept consistent by the subject during the experimental period.

At 120 days after the start of consumption average values for the blood glucose levels and insulin use were calculated as described in 2) and 3) above. Glycated haemoglobin (HbA1c) was determined by the subject's physician as per standard pathology for the 6 months preceding and 3 months after commencing sugarcane fibre consumption.

It was found that average blood glucose levels for breakfast and lunch decreased by 10% and 24.5% respectively after consumption of sugarcane fibre prepared according to the present invention. This corresponds with a drop of almost 28% of insulin required over the same period. During this time insulin use in the evenings and before sleep remained constant. Variation in readings was consistent before and after consumption of sugarcane fibre.

HbA1c data from the subject indicated a significant drop in these levels with the consumption of the sugarcane fibre. The subject's physician indicated that this level of improvement must be the result of a marked improvement in blood glucose control. As the subject did not significantly change food consumption or exercise this indicated sugarcane fibre according to the invention played a significant role in the result.

The subject reported that the sugarcane fibre increased feelings of wellbeing and resulted in moderate weight loss over the test period. The subject also indicated that previous to consumption of sugarcane fibre he had tried a number of diet and exercise regimens to control blood glucose without success. The subject said they felt sure that sugarcane fibre was the source of improvement in pathology. The subject's physician concurred with this assessment.

While Type 2 diabetes mellitus is a condition that has many factors, and as such small scale results cannot always be extrapolated to the general population, however the improvement seen in this example is so marked that these results suggest significant benefit in the consumption of sugarcane fibre according to the invention.

Oral medications used for Type 2 diabetes mellitus rarely report an improvement reduction of HbA1c values greater than 1 point nor do the often result in HbA1c levels below 7. For this reason sugarcane fibre according to the invention should be considered as a suitable adjunct therapy for this condition 2.

TABLE 5
Average values of blood glucose levels and insulin use before and after
the addition of sugarcane fibre to the diet of the test subject.

| | Breakfast Reading | | | Lunch Reading | | |
|---|---|---|---|---|---|---|
| | Ave | StDev | CoV | Ave | StDev | CoV |
| Before SF | 8.8 | 2.2 | 25.7% | 10.5 | 3.6 | 34.2% |
| After SF | 7.9 | 2.1 | 27.2% | 7.9 | 3.4 | 42.5% |
| Relative | 90.0% | | | 75.5% | | |
| After/Before | | | | | | |

| | Breakfast Insulin | | | Lunch Insulin | | |
|---|---|---|---|---|---|---|
| | (units) | | | (units) | | |
| | Ave | StDev | CoV | Ave | StDev | CoV |
| Before SF | 38 | 0.0 | 0.0% | 38 | 0.0 | 0.0% |
| After SF | 27.6 | 1.5 | 5.6% | 27.5 | 1.2 | 4.3% |
| Relative | 72.6% | | | 72.3% | | |
| After/Before | | | | | | |

### Example 8

A comparison was made of reduction of blood glucose levels by sugarcane and a commercially available fibre when taken with fruit juice. The aims of the study were to also determine if addition of sugarcane fibre according to the invention to fruit juice can effectively reduce the GI of the juice, and further to confirm that the addition of pectin to said sugarcane fibre does not interfere with the GI lowering effect.

The GI measurement was adapted from Sydney University G.I. standard methodology. The test was performed in the morning before exercise or food consumption, however alcohol was consumed the previous evening.

Glucose (50 g) was dissolved into water (250 mL) and used as a control.

Test Meals were made from a multi-fruit juice (extra juicy multi fruit with antioxidants; 431.5 mL—equalling 50 g useable carbohydrate) either alone with 4.1 g sugarcane fibre (one serve) or 7 g of a commercially available soluble fibre (Benefibre derived from wheat but claiming to be gluten free; 2 serves). Each meal was consumed in less than 10 minutes.

Blood glucose levels were measured using an Accu-Chek commercial diabetes glucose monitor and provided strips (strips batch number 698 expiry date 30 Apr. 2013). Baseline BGL was taken immediately prior to consuming the meal (t=0) by finger prick using the included lancing device. BGL levels were measured using the above method at t=15, 30, 45, 60, 90, and 120 minutes.

When multiple measurements were taken at the same time point (droplet used, wipe clean and then new droplet used) to determine the error range for the device.

Area under the curve was calculated using the trapezoidal method in Excel. Internal CoV for all of the tests was less than 5%. Juice GI was calculated at 84.5, which is considered high (above 70). Addition of the soluble fibre Benefibre reduced the GI of the juice to 66.7 which is the top of the medium range (55-69).

The addition of 4.1 g sugarcane fibre to the juice reduced the GI to 48.1 which is well in the low range (less than 55). Some systems use a low range of 45-50 which would mean that the addition of sugarcane fibre to juice almost (or does) meet the requirements for low GI in these scales as well.

Even though sugarcane fibre is technically classed as an insoluble fibre, which has previously been thought to have little or no effect on the GI of foods, addition of sugarcane fibre prepared according to the invention to a high GI drink has resulted in a significant reduction of the GI. The reduction in GI was significantly more pronounced than that of a commercially available soluble fibre from wheat.

TABLE 5
Measured blood glucose levels at various times after consumption
of 50 g glucose, 431.5 mL MultiJuice fruit juice +
4.1 g sugarcane fibre or MultiJuice + 7 g Benefibre.

| Time | | | Juice + | Juice + |
|---|---|---|---|---|
| (min) | Glucose | Juice | Benefibre | SF |
| 0 | 0.00 | 0 | 0.00 | 0.00 |
| 15 | 2.48 | 1.65 | 2.50 | 1.80 |
| 30 | 4.88 | 3.35 | 4.05 | 3.40 |
| 45 | 5.32 | 4 | 3.80 | 2.55 |
| 60 | 3.50 | 3 | 1.75 | 1.55 |
| 90 | 0.47 | 1.25 | −0.10 | −0.40 |
| 120 | −0.40 | −0.4 | −0.45 | −0.40 |

TABLE 6
Area under the curve and GI calculations for the increase in blood
glucose levels after consumption of 50 g glucose, 431.5 mL MultiJuice
fruit juice + 4.1 g sugarcane fibre or MultiJuice + 7 g Benefibre.

| Time | | | Juice + | Juice + |
|---|---|---|---|---|
| (min) | Glucose | Juice | Benefibre | SF |
| 0 | 18.63 | 12.375 | 18.75 | 13.50 |
| 15 | 55.25 | 37.5 | 49.13 | 39.00 |
| 30 | 76.50 | 55.125 | 58.88 | 44.63 |
| 45 | 66.13 | 52.5 | 41.63 | 30.75 |
| 60 | 59.50 | 63.75 | 24.75 | 17.25 |
| 90 | 1.00 | 12.75 | −8.25 | −12.00 |
| 120 | 277.00 | 234 | 184.88 | 133.13 |
| GI | 100.0 | 84.5 | 66.7 | 48.1 |

When incorporated in an existing food product format, such as in the examples above, the fibre product described above potentially provides the following benefits to the overall food product:
Tends to increase the fibre content of the food;
May acts as a bulking agent, increasing yield;
Adds moisture to drier formulations (as is commonly the case with gluten-free recipes);
Is allergen-free; and
Tends to reduce the Glycaemic Index (GI) of food to which it is added.

In an alternative form, as per example 5 above, the fibre product can be sold as a stand-alone additive that can be used in conjunction with the individual's normal diet, which allows the following advantages:
Can be sold in single or multiple serve packaging for convenience of use;
May be prepared as a drink (juice or milk based), pudding, or individual sachet;
The dosage can be varied as required for individuals with particularly sensitive digestive systems;
May be taken with normal meals to aid digestion;
May be a combined with other natural products to enhance the digestive health properties of the individual's diet; and
Tends to reduce the GI of any food with which it is combined.

The supplement tends to provide several benefits to the individual, including increasing the fibre content of the food, having a positive effect on digestion, prolonged control of blood glucose levels and lower blood lipid levels.

It will be appreciated by persons skilled in the art that the above described embodiments are not the only ways in which the invention can be put into practice. There are other alternative embodiments which, while different in some details, nevertheless fall within the scope of the invention.

1-12. (canceled)

13. A method of treating an individual's inability to regulate blood glucose levels, said method comprising:
feeding to said individual a food product incorporating dietary fibre material extracted from whole sugar cane wherein a whole sugar cane fibre is prepared by a process including:
(a) a whole sugar cane reduction step;
(b) an aqueous extraction step to separate dietary fibre material from the sugar cane, wherein the pH of the extraction liquid is held between 6.5 and 7.5;
(c) rapid, low-heat drying process so as to enhance the water retention properties of the food product; and
(d) wherein the aqueous extraction step is carried out at a temperature in the range of 25° C. to 70° C.

14. The method of treatment of claim 13, wherein the food product is in the form of a dry powder that can be added to other food products.

15. The method of treatment of claim 13, wherein the food product is in the form of granules.

16. The method treatment of claim 13, wherein the sugar cane fibre material has undergone a pressure heating step at a pressure in the range of 100 psi to 140 psi.

17. A method of treating an individual's inability to regulate blood glucose levels, said method comprising:
feeding to said individual a food product incorporating dietary fibre material extracted from whole sugar cane prepared by a process including: (a) a pressure heating step at a pressure in the range of 100 psi to 140 psi; (b) a whole sugar cane reduction step; (c) an aqueous extraction step to separate dietary fibre material from the sugar cane, wherein the pH of the extraction liquid is held between 6.5 and 7.5; and (d) rapid, low-heat drying of the dietary fibre material that minimizes degradation of the fibre so as to enhance the water retention properties of the food product; and (e) wherein the aqueous extraction step is carried out at a temperature in the range of 25° C. to 70° C.

18. A method of reducing the glycemic index (GI) of a food, said method comprising:
providing a dietary fibre material extracted from whole sugar;
processing said dietary fibre material including: (a) a pressure heating step at a pressure in the range of 100 psi to 140 psi; (b) a whole sugar cane size reduction step; (c) an aqueous extraction step to separate dietary fibre material from the sugar cane, wherein the pH of the extraction liquid is held between 6.5 and 7.5; and (d) rapid, low-heat drying of the dietary fibre material that minimizes degradation of the fibre, and so as to enhance the water retention properties of the food product; and (e) wherein the aqueous extraction step is carried out at a temperature in the range of 25° C. to 70° C.

19. The method of treatment of claim 13, wherein:
said feeding step further includes feeding at least 4 g of said processed dietary fibre material to the individual for at least 120 days resulting in reduction of average blood glucose levels after said 120 days of at least 10% as blood glucose is measured at a breakfast meal.

20. The method of treatment of claim 17, wherein:
said feeding step further includes feeding at least 4 g of said processed dietary fibre material to the individual for at least 120 days resulting in reduction of average blood glucose levels after said 120 days of at least 10% as blood glucose is measured at a breakfast meal.
